(19) United States
(12) Patent Application Publication
(10) Pub. No.: US 2007/0282277 A1
(43) Pub. Date: Dec. 6, 2007

(54) Syringe Driver Housing

(76) Inventors: George Gallagher, St. Asaph (GB); Hal Danby, Wallisellen (CH); Paul Cable, Wallisellen (CH)

Correspondence Address: CHARLES N. QUINN;FOX ROTHSCHILD LLP, 2000 MARKET STREET, 10TH FLOOR, PHILADELPHIA, PA, 19103, US

(21) Appl. No.: 10/582,318

(22) PCT Filed: Dec. 8, 2004
(86) PCT No.: PCT/GB04/05195

§ 371 Date: Apr. 30, 2007

(30) Foreign Application Priority Data
Dec. 9, 2003 (GB) 0328556.6
Jun. 29, 2004 (GB) 0414480.4

(51) Int. Cl. A61M 5/178; A61M 5/315

(52) U.S. Cl. 604/218; 604/214

(57) ABSTRACT

A syringe driver housing (2) for housing syringes of different sizes, the housing comprising a main body part (4) and at least two interchangeable covers (6b) of different sizes. The larger cover is provided with one end (202) adapted to engage an end of the main body part to provide an extension thereto.

The present invention relates to an improved syringe driver housing.

Syringe drivers are well known. They are small, light weight, battery operated machines that are designed to administer subcutaneous infusions of a prescribed amount over a given period. A syringe driver basically consists of the machine itself, a syringe containing the medicine to be administered which is attached to the machine and a thin piece of tubing attached to the syringe which has a needle at the end of it. Syringe drivers are often provided with both the machine and the syringe contained within a housing to increase the portability of the device.

These syringe driver assemblies are provided in different sizes to accommodate syringes of different volumes, such as 5, 10, 20 and 30 ml syringes. The requirement for separate housings to contain syringes of different volumes increases the cost of supplying the syringe drivers since different tooling is required to produce drivers of various sizes. It is desirable to provide a housing for a syringe driver that is suitable for all volumes of syringe.

It is an object of the present invention to provide a syringe driver housing that can accommodate more than one size of syringe.

Accordingly, the present invention provides a housing for a syringe driver assembly comprising a main body part for supporting a syringe and at least two interchangeable covers for attachment to the body part, the covers being of a different size.

The housing may be provided as a kit of parts.

In a preferred embodiment of the present invention, the main body part is sized to receive a syringe of a relatively small length and/or diameter and one of the covers is of a similar length and/or width for extending over the syringe to provide a closed housing. At least one further cover is provided that is of a greater length and/or width for extending over a syringe of a greater length and/or diameter and has one end adapted to engage with an end of the main body part thereby providing an extension to the main body part that is formed integrally with the cover.

It is to be appreciated that any number of different sized covers may be provided for housing syringes of different lengths and/or diameters. Alternatively, just two could be provided that correspond to the smallest and largest syringes that would be contained within the housing.

Preferably, the main body part comprises a container for receiving working parts of the syringe driver with a wall of the container forming a support for receiving the syringe. The main body part is preferably hollow for receiving the working components, such as the motor, battery and electronic controls. A bracket or clip may be provided for retaining the syringe on the support. The main body part may be any desired shape for receiving the component parts.

In a preferred embodiment, each larger cover that provides an extension to the body part has a longitudinal cross-section that is in the general form of a hook comprising a long arm and a tail wherein the long and of the hook forms the cover for laying over the syringe and the tail of the hook forms the extension for mating with the main body. Preferably, the long arm comprises two sides connected by a roof section for surround the syringe. Preferably, both sides of the long arm extend around to form the hook that engages with the main body, thereby forming an internal cavity or recess for receiving the end of the syringe. It is to be appreciated that the long arm of the hook corresponds substantially in length to the extended length of the syringe. Suitable means may be provided for ensuring a tight connection between the tail and the main body.

In an alternative embodiment of the present invention, a separate extension may be attached to a modular body part to increase the length and/or width of the body part thereby enabling the body part to house a syringe of greater dimensions than that housed by a body part that has no extension attached thereto. The main body may have a wall extending from an edge thereof wherein the syringe sits in the recess formed by the container and side wall. More preferably, the main body comprises a rectangular prism having four side faces and two end faces with the side wall extending substantially perpendicularly from one edge thereof to define a L-shaped recess for receiving a syringe. More preferably, a flange or lip extends inwardly from the side wall at one end thereof.

It is preferable for the region of the main body to which the extension may be attached to be provided with a male or female member for mating with a complimentary female or male member provided on the extension. Preferably, one end face of the main body is provided with a male or female member. In a preferred embodiment, the main body part is a moulded component having a male member formed as all integral part thereof.

The extension is constructed such that it will attach to the main body part and serve to extend the region that receives the syringe, such as the L-shaped recess thereby enabling the housing to receive a syringe of greater size than could be received by the main body only. Preferably, the extension has a hollow part that is generally in the form of a sector in plan view, with the straight sides of the sector attaching to the main body part and the arc of the sector forming the end of the housing. In this embodiment, the straight sides preferably define a recess within the extension for receiving an end face of the main body and the arc forms a closed end. An edge of one face of the extension formed by the straight sides is has a side wall extending substantially perpendicularly therefrom for mating with, or abutting, the side wall of the main body part.

At least two different covers are provided that correspond in length and/or width to the length and/or width of the main body part and the length and/or width of the extended main body part respectively. Preferably, the cover is in the form of a substantially L-shaped member that extends over the L-shaped recess of the housing to provide an internal cavity for receiving a syringe. Preferably, the cover is hingedly mounted to the side wall of the main body and/or side wall of the extension.

Preferably, the main body part, covers and extension are formed as moulded components.

For a better understanding of the present invention and to show more clearly how it may be carried into effect, reference will now be made by way of example only to the accompanying drawings in which:—

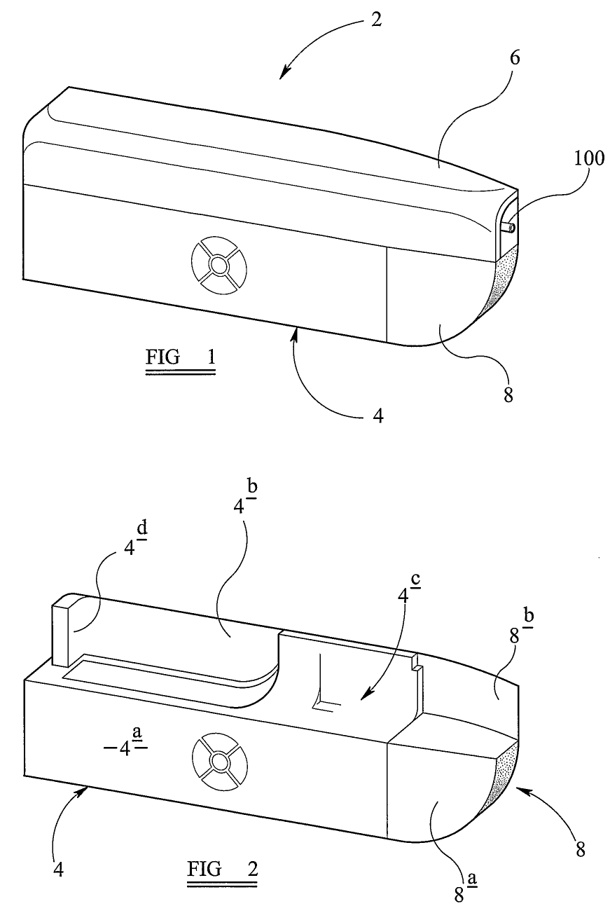

FIG. 1 is a perspective side view of a syringe driver housing according to one embodiment of the present invention, shown adapted for receiving a large syringe;

FIG. 2 is a perspective side view of the syringe driver housing shown in FIG. 1, shown with the cover removed;

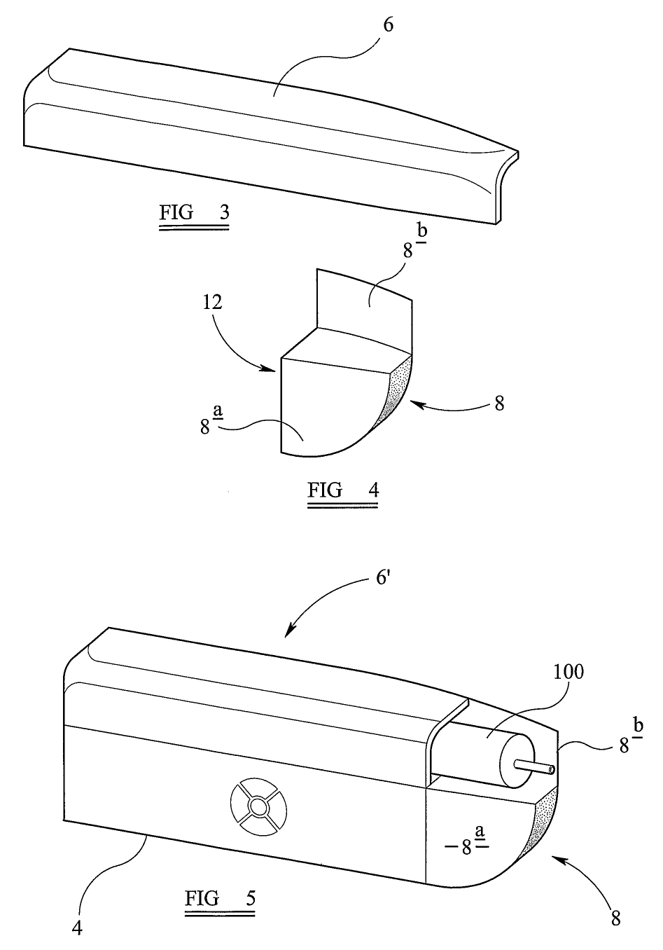

FIG. 3 is a perspective side view of the cover of the syringe driver housing shown in FIG. 1;

FIG. 4 is a perspective side view of the extension that has been removed from the housing shown in FIGS. 1 and 2;

FIG. 5 is a perspective side view of the syringe driver housing of FIG. 1 but having a cover for a small syringe attached thereto;

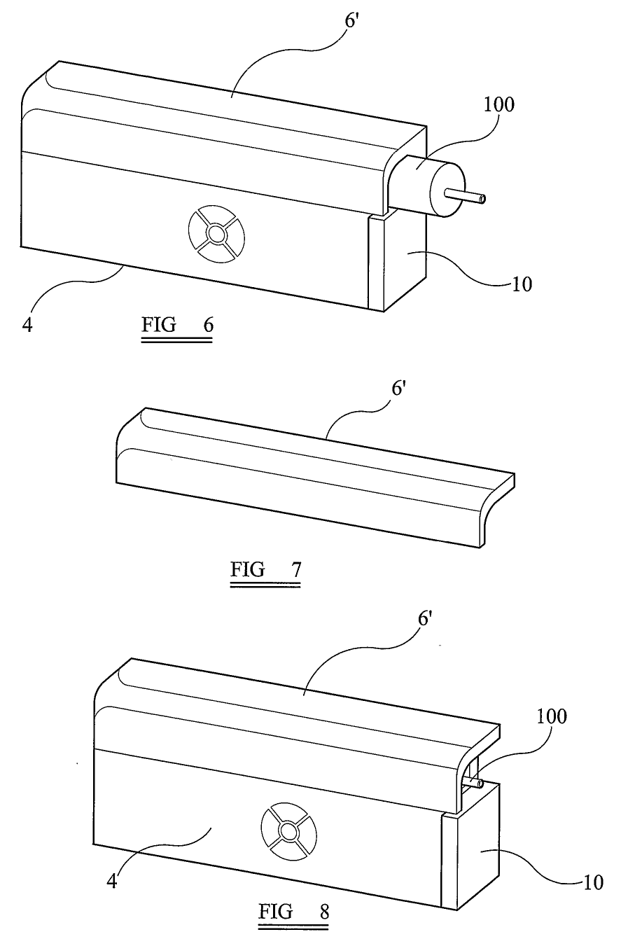

FIG. 6 is a perspective side view of the syringe driver housing shown in FIG. 5 having the front extension removed;

FIG. 7 is a perspective side view of the cover attached to the syringe driver housing shown in FIG. 6;

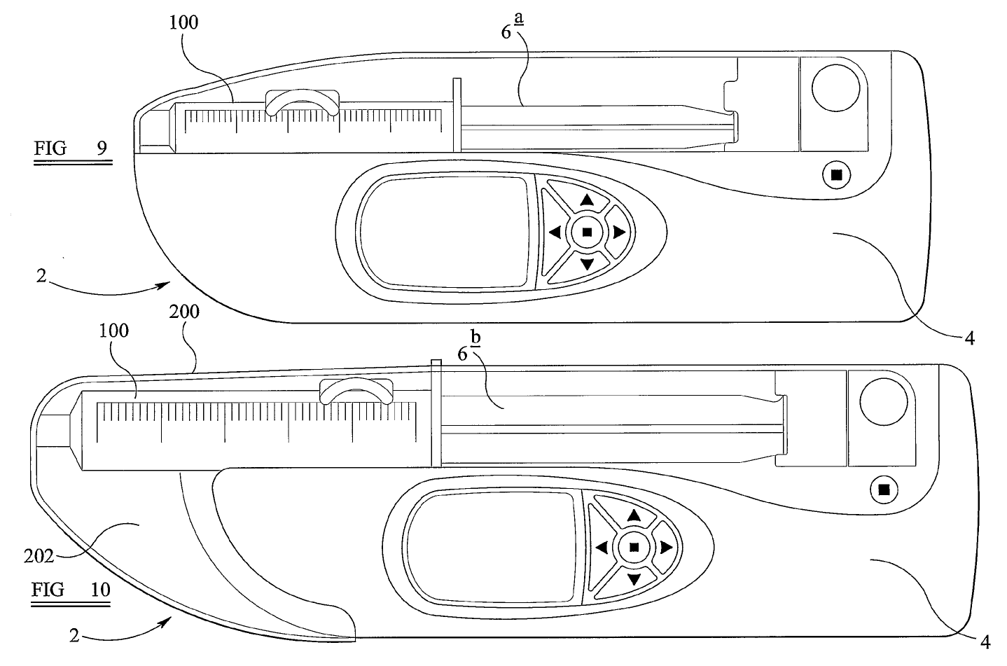

FIG. 8 is a perspective side view of the syringe driver housing according to one embodiment of the present invention, shown adapted for receiving a small syringe;

FIG. 9 is a side view of a syringe driver housing according to an alternative embodiment of the present invention, shown adapted for receiving a small syringe; and

FIG. 10 is a side view of the syringe driver housing shown in FIG. 9, adapted for receiving a large syringe.

Referring to the accompanying drawings, a syringe driver assembly is illustrated that has been modified according to the present invention to enable the syringe housing 2 to accept syringes of different volumes, such as 5, 10, 20 and 30 ml syringes. The invention not only enables syringes of different sizes to be housed within the device but also allows for a reduction in size of the housing when a smaller syringe is to be housed therein, thereby keeping the assembly as small as possible. This is desirable as it results in the device being discrete for the user and fully portable.

FIGS. 1 to 5 of the accompanying drawings illustrate a syringe housing according to one embodiment of the present invention for receiving a relatively large volume syringe, such as 30 ml. The syringe housing 2 comprises a main body 4 for receiving the internal components of the syringe driver or pump, a cover 6 and an extension 8. The internal working components that allow effective operation of the driver (such as the motor, battery and electronic circuitry) are omitted from the drawings for the sake of simplicity. The main body 2 is comprised of a moulded component having a rectangular hollow part 4a for receiving the working components, with one face of the rectangular part having a side wall 4b extending substantially perpendicularly therefrom to define a L-shaped recess 4c for receiving a syringe. A flange 4d extends inwardly at one end of the side wall 4b to act as a stop.

The extension 8 is in the form of a moulded component that is attachable to one end of the main body, preferably by means of a snap-fit connection, and is constructed such as to increase the length of the recess 4c for receiving a long syringe. In the illustrated embodiment, the extension has a main body 8a with a side wall 8b extending substantially perpendicularly from one side thereof, the main body being cone-shaped in plan view to provide a housing that is aesthetically pleasing. However, it is to be appreciated that the extension 8 is not limited to this design. For example, the extension could be in the form of a miniature version of the main body part 4 provided that the extension serves to extend the length of the L-shaped recess for receiving a syringe.

The housing is also provided with a L-shaped cover 6 that is hingedly mounted with respect to the side walls 4b and 8b, being dimensioned to shield the L-shaped recess thereby defining an internal cavity for receiving a syringe 100.

FIGS. 5 to 8 of the accompanying drawings illustrate how the syringe driver housing shown in FIGS. 1 to 4 can be adapted to receive a smaller syringe, such as a 10 ml syringe. The cover 6 is removed from the housing and is replaced with a substantially identical, but smaller, cover 6′ (see FIG. 5). The extension 8 is also removed from the end of the main body 4 to provide a shorter housing for receiving the smaller syringe. FIG. 6 shows the shorter housing containing the relatively large syringe 100 that fitted in the extended syringe housing of FIGS. 1 to 5 whilst FIG. 8 shows the shorter housing containing a smaller syringe 100 that is completely housed within the casing.

Referring in particular to FIGS. 4,6 and 8 of the accompanying drawings, it can be seen that the extension 8 is able to fit onto the main body 4 by mating the male portion 10 provided at one end of the main body with the complimentary female portion 12 provided by the hollow interior of the extension. No additional securing means is required thereby enabling the syringe housing to be easily and quickly adapted to house syringes of different lengths.

It is to be appreciated that a similar modular system could be provided to house syringes of different diameters, for example by having an elongated extension for mating with a side of the main body and a cover that corresponds in dimensions to the recess thus formed.

The present invention provides a modular housing system for receiving the components of a syringe driver assembly that is capable of being quickly adapted to accommodate syringes of different lengths and/or diameters. The invention provides the additional benefit of always providing a housing that is as small as possible given the dimensions of the syringe contained therein which substantially dictate the overall size of the housing.

FIGS. 9 and 10 of the accompanying drawings illustrate a syringe driver housing according to an alternative embodiment of the present invention. The housing 2 again consists of a main body 4 containing the working components of the syringe driver and a cover 6a or 6b hingedly mounted thereto. FIG. 9 illustrates the housing adapted for receiving a syringe 100 of a relatively small length wherein the length of the cover 6a corresponds to the length of the body part 4. A different cover 6b is provided for providing a housing that can receive a syringe of greater length, as illustrated in FIG. 10. The cover 6b is a moulded component and is in the general shape of a hook wherein the long arm 200 of the hook forms the cover over the syringe and the tail 202 of the hook forms all extension for mating with the end of the main body 4 thereby providing a main body of greater length. It is to be appreciated that the inner edges of the tail of the hook have a profile that correspond to the end of the main body. Suitable means may be provided for providing a tight connection between the tail of the hook and the end of the main body.

1. A housing for a syringe driver assembly comprising a main body part for supporting a syringe and at least two interchangeable covers for attachment to the main body part; the main body part being sized to receive a syringe of a relatively small length, a first interchangeable cover is of a similar length to the main body part for extending over the syringe to provide a closed housing; wherein a second cover provides an extension to the body part, the second cover having a longitudinal cross-section in the general form of a hook having a long arm and a tail; the long arm forming the cover for laying over the syringe and the tail forming the extension for mating with the main body part.

2. A housing as claimed in claim 1 wherein the long arm comprises two sides connected by a roof section.

3. A housing as claimed in claim 2 wherein both sides of the long arm extend around to form the hook thereby forming an internal cavity or recess for receiving the end of the syringe.

4. A housing as claimed in claim 3 wherein each cover is hingedly mountable to the main body part.

5. A housing as claimed in claim 4 wherein the main body part and covers are formed as moulded components.

6. A kit of parts for providing a housing for a syringe driver assembly comprising
a) a main body part for supporting a syringe;
b) at least two interchangeable covers for attachment to the body part;
c) a first cover being of a length similar to the main body part for extending over the syringe to provide a closed housing and
d) a second cover providing an extension to the body part, and having a longitudinal cross-section in the general form of a hook including a long arm and a tail, with the long arm forming a cover for laying over the syringe and the tail forming the extension for mating with the main body part.

7. A housing as claimed in claim 1 wherein each cover is hingedly mountable to the main body part.

8. A housing as claimed in claim 2 wherein each cover is hingedly mountable to the main body part.

9. A housing as claimed in claim 1 wherein the main body part and covers are formed as moulded components.

10. A housing as claimed in claim 2 wherein the main body part and covers are formed as moulded components.

11. A housing as claimed in claim 3 wherein the main body part and covers are formed as moulded components.
